(19) United States
(12) Patent Application Publication
(10) Pub. No.: US 2024/0277321 A1
(43) Pub. Date: Aug. 22, 2024

(54) POP-UP SAMPLE COLLECTION DEVICE AND SYSTEM

(71) Applicant: 3M INNOVATIVE PROPERTIES COMPANY, St. Paul, MN (US)

(72) Inventors: Michael R. Berrigan, Oakdale, MN (US); Alan R. Dombrowski, Woodbury, MN (US); Laura R. Nereng, Woodbury, MN (US); Audrey A. Sherman, Woodbury, MN (US); Brett J. Sitter, Marine on St. Croix, MN (US); Narina Y. Stepanova, Woodbury, MN (US)

(21) Appl. No.: 18/562,751

(22) PCT Filed: May 18, 2022
(86) PCT No.: PCT/IB2022/054633

§ 371 Date: Nov. 20, 2023

Related U.S. Application Data
(60) Provisional application No. 63/201,981, filed on May 21, 2021.

(51) Int. Cl. A61B 10/00; A61B 5/097

(52) U.S. Cl. CPC A61B 10/00; A61B 5/097; A61B 2010/0087

(57) ABSTRACT

A sample collection device includes a planar sheet of material having opposing edges and a first major surface and an opposing second major surface, the planar sheet of material having at least two fold lines. A porous sample collection media has at least three edges and opposing major surfaces, at least one edge is fixed to the first major surface of the planar sheet of material. The planar sheet of material is configured to form an airflow channel by fixing the opposing edges together, the airflow channel extends from a mouthpiece end to an air outlet end. The porous sample collection media substantially occludes the airflow channel between the mouthpiece end and the air outlet end.

The present disclosure relates to a pop-up sample collection device and system, methods of using the sample collection devices and systems, and sample collection kits.

### BACKGROUND

As Covid-19 reached pandemic status, increased availability of diagnostic testing was important to help identify and control the serious illness. This illness has highlighted the need for widespread availability of such diagnostic tests even after this pandemic ends. Diagnostic tests typically require a nasopharyngeal swab involving insertion of a 6-inch-long swab into the back of the nasal passage through one nostril and rotation of the swab for approximately 15 seconds. This process is then repeated with the other nostril. The swab is then inserted into a clean container and sent to a lab for processing. Other nasal swab tests require sampling from the mid-turbinate area of the nasal passage—again sampled from both nostrils. Still others require sampling from the anterior nares in both nostrils. Other diagnostic tests involve collecting a saliva sample and then placing it in a clean container and sending it to a lab for processing. Currently available at-home viral (for example Covid-19) tests involve a nasal swab as a described above but do not require sending to a lab for processing (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

The sample collection devices and test processes described above have various challenges. For example, the available tests typically require that the collection device be processed at a laboratory, increasing cost and delaying delivery of results. Further, many of the test methods require that the sample collection mechanism be a nasopharyngeal or other type of nasal or oral swab, which is uncomfortable for the user. This discomfort can cause users to opt out of testing. Further, there may be possibility of contamination of the sample during transfer to the clean container, removal from the container, etc. Due to the multiple steps and devices involved and the possibility of contamination of the sample, such conventional methods and devices for sample collection and eluent testing may be used by only trained professionals (e.g., medical personnel), and may be complicated for use by a user with little or no training.

As such, the present disclosure provides easy-to-use, inexpensive sample collection devices that can be used in any location by a layperson. Additionally, the sample collection device described herein does not require the user to undergo a nasopharyngeal or other nasal or oral swab. These sample collection devices may be mailed or delivered flat to a user and then the user may pop-up the sample collection device collect the sample, force the device flat and mail or deliver the sample collection device to a testing location to analyze the test.

There is a need for an inexpensive, simple to use, and reliable sample collection device that may be used by laypeople around the world to test to see if they are shedding pathogens or virus. This sample collection device may be paired with a testing device to determine the presence or absence of pathogen or virus in the collected sample.

A sample collection device includes a planar sheet of material having opposing edges and a first major surface and an opposing second major surface, the planar sheet of material having at least two fold lines. A porous sample collection media has at least three edges and opposing major surfaces, at least one edge is fixed to the first major surface of the planar sheet of material. The planar sheet of material is configured to form an airflow channel by fixing the opposing edges together, the airflow channel extends from a mouthpiece end to an air outlet end. The porous sample collection media substantially occludes the airflow channel between the mouthpiece end and the air outlet end.

The sample collection device may be combined with instructions for collecting a sample to form a kit. The instructions may instruct the user to: form the airflow channel from the planar sheet of material; exhale into the airflow channel to capture a sample in the porous sample collection media; the fold the airflow channel flat; seal the air outlet end; and seal the mouthpiece end.

A method includes: flowing exhalation air into sample collection device described herein, forming a loaded porous sample collection media; folding the airflow channel flat; sealing the air outlet end; and sealing the mouthpiece end.

It may be desirable to provide a system that includes both a sample collector device and rapid antigen testing device. The system may advantageously be self-contained and sterile such that (unlike swabs which may be contaminated upon use and then become more contaminated during testing, increasing background noise when testing) the pathogen or virus detection may be more accurate or reliable.

### BRIEF DESCRIPTION OF FIGURES

FIG. 1 is a front elevation schematic diagram of an illustrative planar sheet of material.

FIG. 2 is a side elevation cross-sectional schematic diagram of the illustrative sample collection device of FIG. 1 flowing exhalation airflow through the sample collection device.

FIG. 3 is a front elevation schematic diagram of the illustrative sample collection device of FIG. 2 folded flat.

FIG. 4 is a transparent perspective schematic diagram of the illustrative sample collection device of FIG. 2.

FIG. 5 is a front elevation schematic diagram of an illustrative planar sheet of material.

FIG. 6 is a side elevation cross-sectional schematic diagram of the illustrative sample collection device of FIG. 5 flowing exhalation airflow through the sample collection device.

FIG. 7 is a front elevation schematic diagram of the illustrative sample collection device of FIG. 6 folded flat.

FIG. 8 is a transparent perspective schematic diagram of the illustrative sample collection device of FIG. 6.

FIG. 9 is a front elevation schematic diagram of an illustrative planar sheet of material.

FIG. 10 is a side elevation cross-sectional schematic diagram of the illustrative sample collection device of FIG. 9 flowing exhalation airflow through the sample collection device.

FIG. 11 is a front elevation schematic diagram of the illustrative sample collection device of FIG. 10 folded flat.

FIG. 12 is a transparent perspective schematic diagram of the illustrative sample collection device of FIG. 10.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

The term “about” is used here in conjunction with numeric values to include normal variations in measurements as expected by persons skilled in the art and is understood have the same meaning as “approximately” and to cover a typical margin of error, such as +5% of the stated value.

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The terms “downstream” and “upstream” refer to a relative position based on a direction of exhalation airflow through the device. For example, the upstream-most element of the device is the mouthpiece element, and the downstream-most element of the device is the air outlet end.

### DETAILED DESCRIPTION

The present disclosure relates to a sample collection device, kit, system, and method. The present disclosure relates to a bioaerosol collection device, kit, system, and method.

A sample collection device includes a planar sheet of material having opposing edges and a first major surface and an opposing second major surface, the planar sheet of material having at least two fold lines. A porous sample collection media has at least three edges and opposing major surfaces, at least one edge is fixed to the first major surface of the planar sheet of material. The planar sheet of material is configured to form an airflow channel by fixing the opposing edges together, the airflow channel extends from a mouthpiece end to an air outlet end. The porous sample collection media substantially occludes the airflow channel between the mouthpiece end and the air outlet end.

As used herein, the term “mouthpiece” is not limited to exhalation from the mouth. Instead, the term “mouthpieces” merely refers to an exhalation receipt portion or device that is capable of receiving exhalation by the user from, for example, the user's mouth or nose.

While the porous sample collection media is illustrated herein as defining a planar element, it is understood that the porous sample collection media may define any shape when disposed within the housing and along the airflow channel.

The sample collection device includes a porous sample collection media disposed within the device housing and along an airflow channel defined by the device housing. Once a sample is collected on the porous sample collection media the sample collection device may be folded flat and may be sealed for testing at a future time and/or remote location to the sample collection location.

The sample collection device may be received by the user assembled but already folded flat. The user may pop-up the folded flat sample collection device and exhale into the airflow channel defined by the device housing to collect a sample on the porous sample collection media. The user may then fold the sample collection device flat and optionally seal or close the mouthpiece end and the air outlet end to preserve the sample within the sample collection device for testing at a future time and/or remote location to the sample collection location.

The planar sheet of material is impervious to fluid. The planar sheet of material is impervious to liquid. The planar sheet is impervious to fluid or liquid and comprises cellulosic material. The planar sheet of material may be formed of paper or paperboard with a polymeric layer. The planar sheet of material may have a thickness in a range from 0.5 mm to 2 mm, or 0.7 mm to 1 mm, or 0.8 mm to 0.9 mm.

The porous sample collection media is fixed within the airflow channel and spaced apart from each of the air outlet end to the mouthpiece end, to prevent a user from touching and contaminating the porous sample collection media. The housing has a length value L from the mouthpiece end to the air outlet end, and the porous sample collection media is recessed from both the mouthpiece end and the air outlet end at least 20% of the length value L, or at least 25% of the length value L, or at least 30% of the length value L. or at least 35% of the length value L, or in a range from 25% to 50% of the length value L.

The porous sample collection media may be a nonwoven material configured to filter or capture pathogens or virus from an exhalation airflow. The porous sample collection media may be a nonwoven material or filtration layer having an electrostatic charge configured to filter or capture pathogens or virus from an exhalation airflow. The porous sample collection media may be a hydrophobic nonwoven filtration layer or material configured to filter or capture pathogens or virus from an exhalation airflow. The porous sample collection media may be a hydrophobic nonwoven filtration layer or material having an electrostatic charge configured to filter or capture pathogens or virus from an exhalation airflow.

The term “hydrophobic” refers to a material having a water contact angle of 90 degrees or greater, or from about 90 degrees to about 170 degrees, or from about 100 degrees to about 150 degrees. Water contact angle is measured using ASTM D5727-1997 Standard test method for surface wettability and absorbency of sheeted material using an automated contact angle tester.

The porous sample collection media may be formed of polymeric material. The porous sample collection media may be formed of a polyolefin. The porous sample collection media may be formed of polypropylene. One illustrative porous sample collection media is commercially available from 3M Company (St. Paul MN, U.S.A.) under the trade designation FILTRETE Smart MPR 1900 Premium Allergen, Bacteria & Virus Air Filter Merv 12 to 14, or Merv 13.

The porous sample collection media may be formed of a of a polylactide (PLA) such as, for example, 6100D from NatureWorks LLC15305 Minnetonka Blvd Minnetonka, MN 55345. Exemplary nonwoven filtration layer materials for use in or as the porous sample collection media include, for example, those described in U.S. Pat. Nos. 7,947,142; 8,162,153; 9,139,940; and 10,273,612, all of which are incorporated herein in their entirety.

The porous sample collection media may have a thickness (orthogonal to the major plane) in a range from 200 micrometers to 1000 micrometers, or from 250 micrometers to 750 micrometers. The porous sample collection media may have major plane surface area in a range from about 1 cm2 to about 4 cm2, or about 2 cm2 to about 3 cm2, and is sufficient to fill the cross-section of the airflow channel. The airflow channel may have a cross-sectional area in a range from 1 cm2 to about 4 cm2, or about 2 cm2 to about 3 cm2.

In some embodiments, the sample collection device further includes a pre-filter or screen (not shown) disposed in the housing, and upstream of the porous sample collection media to catch solid material or debris (such as, for example, food particles) so that they are not incident on the porous sample collection media. The pre-filter or screen (not shown) may be fixed within the housing and along the airflow channel and between the mouthpiece end and the porous sample collection media. In some embodiments, the pre-filter or screen includes one or more flow apertures therethrough. The exhalation airflow passes through a thickness of the pre-filter or screen. The pre-filter or screen at least partially occludes the air flow channel. In some cases, the pre-filter or screen may have a major plane (not shown) that is orthogonal to the direction of the exhalation airflow (not shown) passing through the thickness of the pre-filter or screen. The pre-filter or screen may be a non-woven layer configured to filter out or capture larger particles from the exhalation airflow passing through the pre-filter or screen. In some cases, the pre-filter or screen may be a non-woven layer that does not have an electrostatic charge. In some embodiments, the pre-filter or screen does not filter or capture significant amounts of viral or pathogen material and instead allows them to transmit through the pre-filter or screen. In some embodiments, the pre-filter or screen is made of or includes at least one of a plastic mesh, a woven net, a needle tacked fibrous web, a knitted mesh, an extruded net, and/or a carded or spunbond coverstock.

The porous sample collection media may be fixed within the housing and along the airflow channel. Exhalation airflow passes through the thickness of the porous sample collection media. The sample collection device is configured to allow a user to breath comfortably into the sample collection device with minimal pressure drop across the porous sample collection media. For example, a user may exhale into the sample collection device at a rate of less than 85 liters/min, or less than 50 liters/minute, or 35 liters/min, or less than 24 liters/min, or less than 10 liters/min and experience a pressure drop across the porous sample collection media of 70 mm water or less, or 50 mm of water or less, or 25 mm or water or less, or 10 mm of water or less, or 5 mm of water or less.

The planar sheet of material may include an adhesive configured to fix opposing edges together to form the airflow channel of the sample collection device. The adhesive may be disposed on a first or second major surface of the planar sheet and form a continuous adhesive strip co-extensive with the one or both of the opposing edges of the planar sheet of material. The adhesive may be a pressure sensitive adhesive. The adhesive may be covered with a release liner. The user may remove the release liner to expose the adhesive and form the airflow channel of the sample collection device.

The planar sheet of material may include an adhesive configured to fix opposing edges together to seal the mouthpiece end and the air outlet end and seal the airflow channel of the sample collection device following collection of the sample by the user. The adhesive may be disposed on a first or second major surface of the planar sheet and form a continuous adhesive strip co-extensive with the inner surface defining the mouthpiece end and the air outlet end. The adhesive may be a pressure sensitive adhesive. The adhesive may be covered with a release liner. The user may remove the release liner to expose the adhesive and form the airflow channel of the sample collection device.

In some embodiments, the sample collection device further includes a mouthpiece conduit configured to interact with the mouthpiece end of the sample collection device. The mouthpiece conduit may be a separate element from the planar sheet of material and may be included in a kit or sample collection system.

The disclosure is also directed to a sample collection system. The sample collection system includes the sample collection device herein and an assay configured to receive liquid from the sample collection device. The assay may be a separate element from the sample collection device. The assay ay be configured to receive liquid from the sample collection device.

The assay may be a flow assay, such as a lateral flow assay or a vertical flow assay. The assay may detect a virus or pathogen. The assay may include a test result display window to indicate the presence or absence of a virus or pathogen.

An assay may be referred to as lateral flow assays (LFAs) or vertical flow assays (VFAs) which are, generally, paper-based platforms for the detection and quantification of analytes in complex mixtures, where the sample is placed on a test device and the results are displayed within 5-30 min. Low development costs and ease of production of LFAs have resulted in the expansion of its applications to multiple fields in which rapid tests are required. LFA-based tests are widely used in hospitals, physician's offices and clinical laboratories for the qualitative and quantitative detection of specific antigens and antibodies, as well as products of gene amplification. A variety of biological samples can be tested using assays.

The disclosure is also directed to a kit including the sample collection device described above, and instructions for collecting a sample. The kit may be a package provided to a user to complete the sample collection process.

The kit instructions may include instructions to:
form the airflow channel from the planar sheet of material;
exhale into the airflow channel of the housing to capture a sample in the porous sample collection media;
fold the airflow channel flat;
seal the air outlet end; and
seal the mouthpiece end.

According to another embodiment, a method includes flowing exhalation air through a porous sample collection media. The porous sample collection media is disposed in an airflow channel forming a loaded porous sample collection media. Then folding the airflow channel flat. Then sealing the air outlet end and the mouthpiece end.

The method may include flowing liquid through the loaded porous sample collection media, forming an eluent, and then testing the elute for a virus or pathogen presence.

The method may further include, testing the eluent with an assay. The testing may include testing the eluent for a virus or pathogen presence. The method may include flowing a metered dose in a range from 50 microliters to 400 microliters of liquid through the loaded porous sample collection media disposed in the airflow channel. The method may include flowing a metered dose of aqueous liquid comprising a surfactant through the loaded porous sample collection media disposed in the airflow channel. The method may include squeezing the housing, air outlet end cap, or mouthpiece end cap to urge eluent through a nozzle or liquid dropper on the mouthpiece end cap.

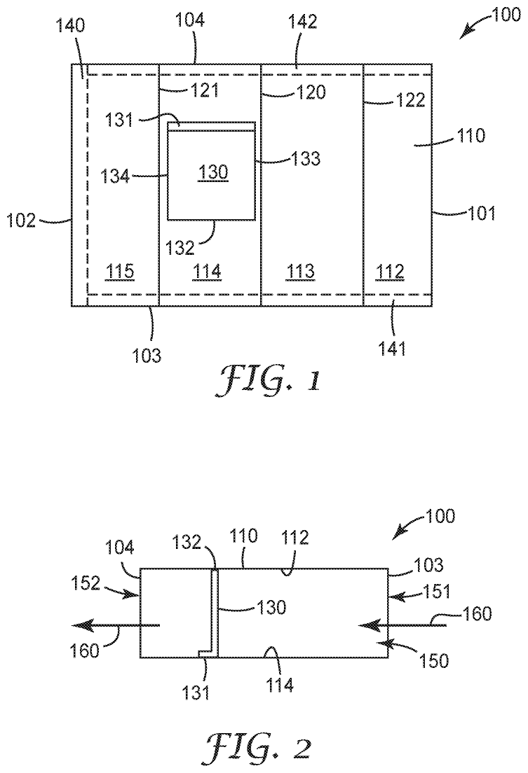

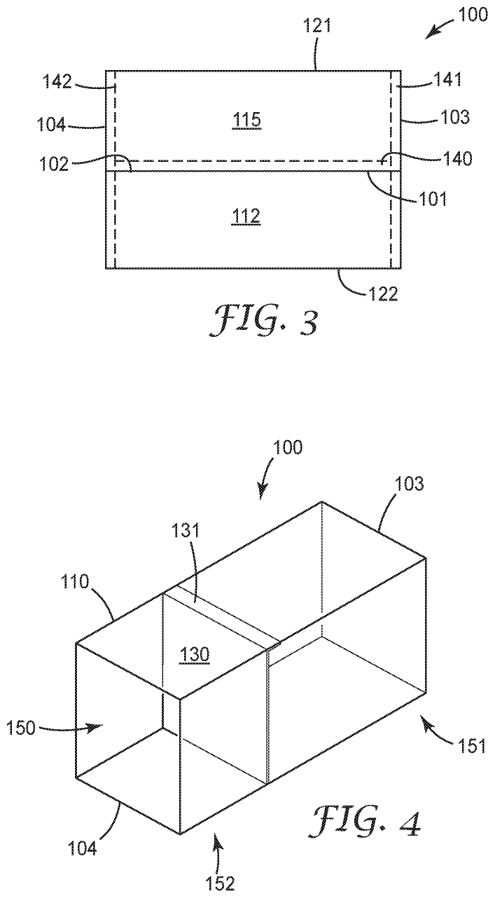

FIG. 1 is a front elevation schematic diagram of an illustrative planar sheet of material 100. FIG. 2 is a side elevation cross-sectional schematic diagram of the illustrative sample collection device 100 of FIG. 1 flowing exhalation airflow 160 through the sample collection device 100. FIG. 3 is a front elevation schematic diagram of the illustrative sample collection device 100 of FIG. 2 folded flat. FIG. 4 is a transparent perspective schematic diagram of the illustrative sample collection device 100 of FIG. 2.

The sample collection device 100 includes a planar sheet of material 110 has opposing edges 101, 102 separated second opposing edges 103, 104. Opposing edges 101, 102 may be parallel with each other. Second opposing edges 103, 104 may be parallel with each other. Opposing edges 101, 102 may be orthogonal to the second opposing edges 103, 104. The planar sheet of material 110 has a first major surface and an opposing second major surface. The first major surface and an opposing second major surface may be parallel with each other.

The planar sheet of material 110 has at least two fold lines 120, 121, 122. A porous sample collection media 130 has at least three edges 131, 132, 133, 134 and opposing major surfaces. At least one edge 131 is fixed to the first major surface of the planar sheet of material 110. The planar sheet of material 110 is configured to form an airflow channel 150 by fixing the opposing edges 101, 102 together. The airflow channel 150 extends from a mouthpiece end 151 to an air outlet end 152. The porous sample collection media 130 substantially occludes the airflow channel 150 between the mouthpiece end 151 and the air outlet end 152. Exhalation airflow 160 flows from the mouthpiece end 151 through the porous sample collection media 130 and exits through the air outlet end 152.

The planar sheet of material 110 forms a rectangle and the porous sample collection media 130 forms a rectangle and a single edge 131 of the porous sample collection media 130 is fixed to the first major surface of the planar sheet of material 110. The planar sheet of material 110 includes parallel fold lines 120, 121, 122 that define sheet panels 112, 113, 114, 115. The parallel fold lines 120, 121, 122 may be parallel to the opposing edges 101, 102.

The planar sheet of material 110 includes fold lines 120, 121 parallel with and adjacent to opposing sides 133, 134 of the porous sample collection media 130 that are orthogonal to the fixed edge 131.

The planar sheet of material 110 may include an adhesive strip 140 configured to fix the opposing edges 101, 102 together to form the airflow channel 150. The adhesive strip 140 may form a continuous element coextensive with the at least one of the opposing edges 101, 102.

The planar sheet of material 110 may include a mouthpiece end 151 adhesive strip 141 configured to seal the mouthpiece end 151 of the airflow channel 150 once the sample is collected. The adhesive strip 141 may form a continuous element coextensive with the mouthpiece end 151.

The planar sheet of material 110 may include an air outlet end 152 adhesive strip 142 configured to seal the air outlet end 152 of the airflow channel 150 once the sample is collected. The adhesive strip 142 may form a continuous element coextensive with the air outlet end 152.

The mouthpiece end 151 of the sample collection device 100 may folded back into the airflow channel 150 to close off the mouthpiece end 151 and secure the porous sample collection media 130 once a sample has been collected. The air outlet end 152 of the sample collection device 100 may folded back into the airflow channel 150 to close off the air outlet end 152 and secure the porous sample collection media 130 once a sample has been collected.

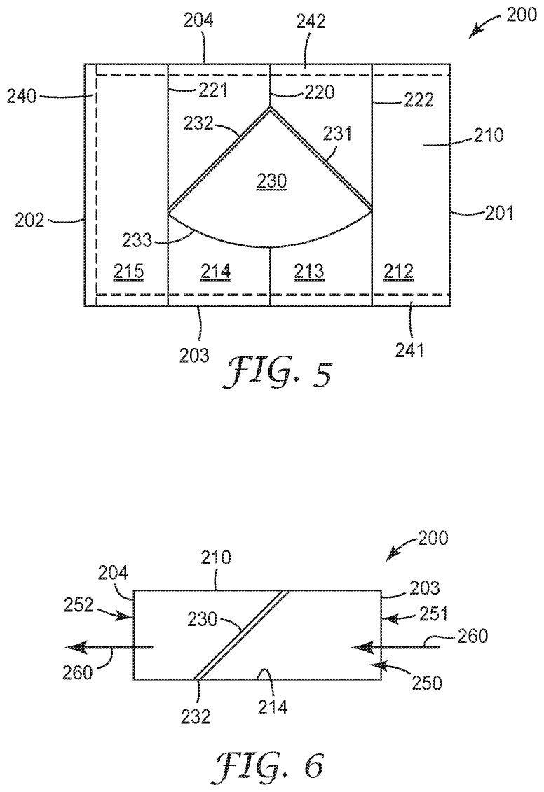

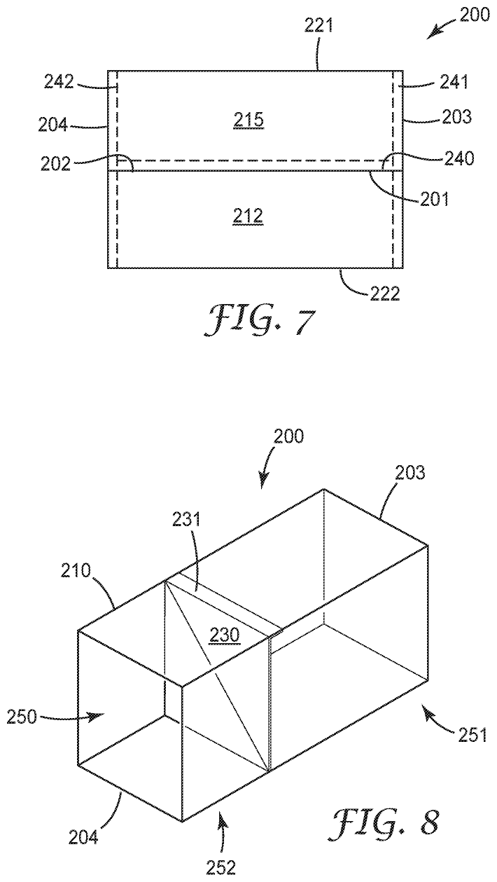

FIG. 5 is a front elevation schematic diagram of an illustrative planar sheet of material 200. FIG. 6 is a side elevation cross-sectional schematic diagram of the illustrative sample collection device 200 of FIG. 5 flowing exhalation airflow 260 through the sample collection device 200. FIG. 7 is a front elevation schematic diagram of the illustrative sample collection device 200 of FIG. 6 folded flat. FIG. 8 is a transparent perspective schematic diagram of the illustrative sample collection device 200 of FIG. 6.

The sample collection device 200 includes a planar sheet of material 210 has opposing edges 201, 202 separated second opposing edges 203, 204. Opposing edges 201, 202 may be parallel with each other. Second opposing edges 203, 204 may be parallel with each other. Opposing edges 201, 202 may be orthogonal to the second opposing edges 203, 204. The planar sheet of material 210 has a first major surface and an opposing second major surface. The first major surface and an opposing second major surface may be parallel with each other.

The planar sheet of material 210 has at least two fold lines 220, 221, 222. A porous sample collection media 230 has at least three edges 231, 232, 233 and opposing major surfaces. At least one edge 231, 231 is fixed to the first major surface of the planar sheet of material 210. The planar sheet of material 210 is configured to form an airflow channel 250 by fixing the opposing edges 201, 202 together. The airflow channel 250 extends from a mouthpiece end 251 to an air outlet end 252. The porous sample collection media 230 substantially occludes the airflow channel 250 between the mouthpiece end 251 and the air outlet end 252. Exhalation airflow 260 flows from the mouthpiece end 251 through the porous sample collection media 230 and exits through the air outlet end 252.

The planar sheet of material 210 forms a rectangle and the porous sample collection media 230 forms a flat cone shape or polygon and two linear converging edges 231, 232 of the porous sample collection media 230 are fixed to the first major surface of the planar sheet of material 210. The planar sheet of material 210 includes parallel fold lines 220, 221, 222 that define sheet panels 212, 213, 214, 215. The parallel fold lines 220, 221, 222 may be parallel to the opposing edges 201, 202.

The planar sheet of material 210 includes fold line 220 bisecting the porous sample collection media 230 at an apex of the linear converging edges 231, 232 of the porous sample collection media 230 that is parallel with the mouthpiece end 251 and the air outlet end 252. The planar sheet of material 210 includes fold lines 221, 222 that are parallel with the fold line 220.

The planar sheet of material 210 may include an adhesive strip 240 configured to fix the opposing edges 201, 202 together to form the airflow channel 250. The adhesive strip 240 may form a continuous element coextensive with the at least one of the opposing edges 201, 202.

The planar sheet of material 210 may include a mouthpiece end 251 adhesive strip 241 configured to seal the mouthpiece end 251 of the airflow channel 250 once the sample is collected. The adhesive strip 241 may form a continuous element coextensive with the mouthpiece end 251.

The planar sheet of material 210 may include an air outlet end 252 adhesive strip 242 configured to seal the air outlet end 252 of the airflow channel 250 once the sample is collected. The adhesive strip 242 may form a continuous element coextensive with the air outlet end 252.

The mouthpiece end 251 of the sample collection device 200 may folded back into the airflow channel 250 to close off the mouthpiece end 251 and secure the porous sample collection media 230 once a sample has been collected. The air outlet end 252 of the sample collection device 200 may folded back into the airflow channel 250 to close off the air outlet end 252 and secure the porous sample collection media 230 once a sample has been collected.

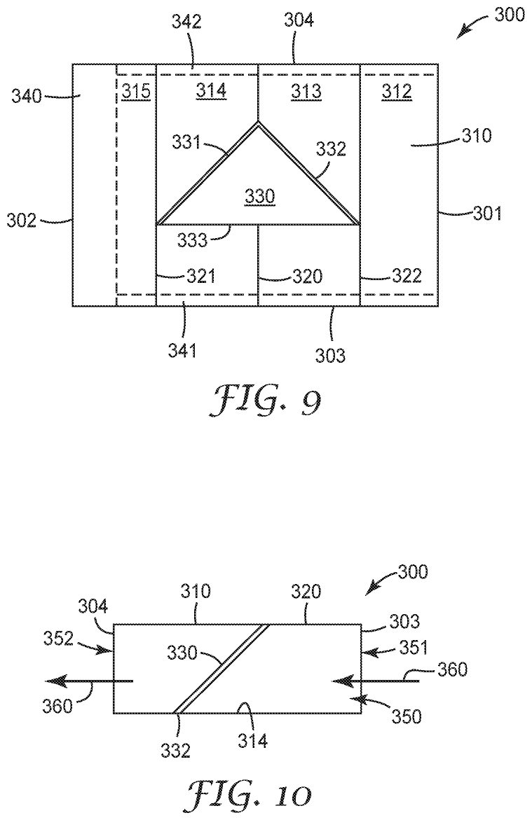

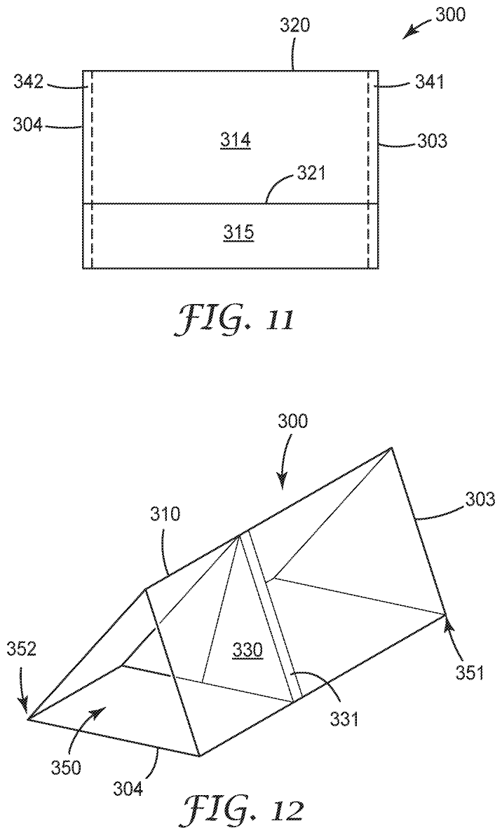

FIG. 9 is a front elevation schematic diagram of an illustrative planar sheet of material 300. FIG. 10 is a side elevation cross-sectional schematic diagram of the illustrative sample collection device 300 of FIG. 9 flowing exhalation airflow 360 through the sample collection device 300. FIG. 11 is a front elevation schematic diagram of the illustrative sample collection device 300 of FIG. 10 folded flat. FIG. 12 is a transparent perspective schematic diagram of the illustrative sample collection device 300 of FIG. 10.

The sample collection device 300 includes a planar sheet of material 310 has opposing edges 301, 302 separated second opposing edges 303, 304. Opposing edges 301, 302 may be parallel with each other. Second opposing edges 303, 304 may be parallel with each other. Opposing edges 301, 302 may be orthogonal to the second opposing edges 303, 304. The planar sheet of material 310 has a first major surface and an opposing second major surface. The first major surface and an opposing second major surface may be parallel with each other.

The planar sheet of material 310 has at least two fold lines 320, 321, 322. A porous sample collection media 330 has at least three edges 331, 332, 333 and opposing major surfaces. At least one edge 331, 331 is fixed to the first major surface of the planar sheet of material 310. The planar sheet of material 310 is configured to form an airflow channel 350 by fixing the opposing edges 301, 302 together. The airflow channel 350 extends from a mouthpiece end 351 to an air outlet end 352. The porous sample collection media 330 substantially occludes the airflow channel 350 between the mouthpiece end 351 and the air outlet end 352. Exhalation airflow 360 flows from the mouthpiece end 351 through the porous sample collection media 330 and exits through the air outlet end 352.

The planar sheet of material 310 forms a rectangle and the porous sample collection media 330 forms a triangle and two edges 331, 332 of the porous sample collection media 330 are fixed to the first major surface of the planar sheet of material 310. The planar sheet of material 310 includes parallel fold lines 320, 321, 322 that define sheet panels 312, 313, 314, 315. The parallel fold lines 320, 321, 322 may be parallel to the opposing edges 301, 302.

The planar sheet of material 310 includes fold line 320 bisecting the porous sample collection media 330 at an apex of the fixed edges 331, 332 of the porous sample collection media 330 that is parallel with the mouthpiece end 351 and the air outlet end 352. The planar sheet of material 310 includes fold lines 321, 322 that are parallel with the fold line 320.

The planar sheet of material 310 may include an adhesive strip 340 configured to fix the opposing edges 301, 302 together to form the airflow channel 350. The adhesive strip 340 may form a continuous element coextensive with the at least one of the opposing edges 301, 302.

The planar sheet of material 310 may include a mouthpiece end 351 adhesive strip 341 configured to seal the mouthpiece end 351 of the airflow channel 350 once the sample is collected. The adhesive strip 341 may form a continuous element coextensive with the mouthpiece end 351.

The planar sheet of material 310 may include an air outlet end 325 adhesive strip 342 configured to seal the air outlet end 352 of the airflow channel 350 once the sample is collected. The adhesive strip 342 may form a continuous element coextensive with the air outlet end 352.

The mouthpiece end 351 of the sample collection device 300 may folded back into the airflow channel 350 to close off the mouthpiece end 351 and secure the porous sample collection media 330 once a sample has been collected. The air outlet end 352 of the sample collection device 300 may folded back into the airflow channel 350 to close off the air outlet end 352 and secure the porous sample collection media 330 once a sample has been collected. The mouthpiece end 351 and the air outlet end 352 of the sample collection device 300 may include two or more flaps 9 not shown) hingedly attached to each of the mouthpiece end 351 and the air outlet end 352 and configured to occlude the mouthpiece end 351 and the air outlet end 352 once the sample is collected.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A sample collection device comprising:
a planar sheet of material having opposing edges and a first major surface and an opposing second major surface, the planar sheet of material having at least two fold lines;
a porous sample collection media having at least three edges and opposing major surfaces, at least one edge is fixed to the first major surface of the planar sheet of material;
wherein the planar sheet of material is configured to form an airflow channel by fixing the opposing edges together, the airflow channel extends from a mouthpiece end to an air outlet end; and
the porous sample collection media substantially occludes the airflow channel between the mouthpiece end and the air outlet end.

2. The sample collection device according to claim 1, wherein the planar sheet of material forms a rectangle and the porous sample collection media forms a rectangle and a single edge of the porous sample collection media is fixed to the first major surface of the planar sheet of material.

3. The sample collection device according to claim 2, wherein the planar sheet of material comprises fold lines parallel with and adjacent to opposing sides of the porous sample collection media that are orthogonal to the fixed edge.

4. The sample collection device according to claim 1, wherein the planar sheet of material forms a rectangle and the porous sample collection media forms a flat cone shape and two linear converging edges of the porous sample collection media are fixed to the first major surface of the planar sheet of material.

5. The sample collection device according to claim 4, wherein the planar sheet of material comprises a fold line bisecting the porous sample collection media at an apex of the flat cone shape.

6. The sample collection device according to claim 1, wherein the planar sheet of material forms a rectangle and the porous sample collection media forms a triangle and two edges of the porous sample collection media are fixed to the first major surface of the planar sheet of material.

7. The sample collection device according to claim 6, wherein the planar sheet of material comprises a fold line bisecting the porous sample collection media at an apex of the two fixed edges of the triangle.

8. The sample collection device according to claim 1, wherein the planar sheet of material forms a rectangle and the porous sample collection media forms a polygon and two edges of the porous sample collection media are fixed to the first major surface of the planar sheet of material.

9. The sample collection device according to claim 8, wherein the planar sheet of material comprises a fold line bisecting the porous sample collection media at an apex of the two fixed edges of the polygon.

10. The sample collection device according to claim 1, wherein the planar sheet of material is impervious to fluid.

11. The sample collection device according to claim 10, wherein the planar sheet of material comprises cellulosic material.

12. The sample collection device according to claim 1, wherein the porous sample collection media comprises a nonwoven filtration layer having an electrostatic charge.

13. The sample collection device according to claim 12, wherein the nonwoven filtration layer is hydrophobic.

14. The sample collection device according to claim 12, wherein the nonwoven filtration layer is formed from polypropylene.

15. The sample collection device according to claim 12, wherein the nonwoven filtration layer is formed from polylactide.

16. The sample collection device according to any of claim 12, wherein the nonwoven filtration layer has a thickness in a range from 200 to 1000 micrometers, or from 250 to 750 micrometers.

17. The sample collection device according to claim 1, wherein the airflow channel has a length value L from the mouthpiece end to the air outlet end, and the porous sample collection media is recessed from both the mouthpiece end and the air outlet end at least 20% of the length value L.

18. The sample collection device according to claim 1, wherein the sample collection device is configured to be folded flat.

19. The sample collection device according to claim 1, wherein the sample collection device is configured to seal the air outlet end and the mouthpiece end once a sample is collected onto the porous sample collection media.

20. The sample collection device according claim 1, further comprising an adhesive material disposed on the air outlet end and the mouthpiece end configured to seal the air outlet end and the mouthpiece end once a sample is collected onto the porous sample collection media.

22-32. (canceled)
